(19) United States
(12) Patent Application Publication
(10) Pub. No.: US 2023/0045584 A1
(43) Pub. Date: Feb. 9, 2023

(54) APPARATUS AND METHOD FOR DETECTING GAS

(71) Applicant: Maschinenfabrik Reinhausen GmbH, Regensburg (DE)

(72) Inventors: Nicolae Barsan, Tuebingen (DE); Juergen Schuebel, Frankfurt am Main (DE); Andreas Kurz, Frankfurt am Main (DE)

(21) Appl. No.: 17/758,805

(22) PCT Filed: Dec. 16, 2020
(86) PCT No.: PCT/EP2020/086393

§ 371 Date: Jul. 14, 2022

(30) Foreign Application Priority Data
Jan. 20, 2020 (DE) 10 2020 101 132.6

(51) Int. Cl. G01N 33/28; G01N 33/00; G01N 30/88

(52) U.S. Cl. CPC G01N 33/2841; G01N 33/0031; G01N 30/88; G01N 2030/8886; G01N 2030/326

(57) ABSTRACT

An apparatus detects gas in a high-voltage device, which is filled with an insulating medium. The apparatus has: an inlet configured for introducing a carrier gas; an outlet configured for discharging the carrier gas; at least one gas sensor configured to detect a gas; a first pump configured to convey the carrier gas in the apparatus; a membrane which comprises at least one semipermeable basic material, which is at least partially surrounded by the insulating medium, and which is arranged to be at least partially subjected to an incident flow of the carrier gas; a second pump configured to convey the carrier gas into the apparatus and out of the apparatus; and a separating column, which is arranged before the gas sensor. The gas sensor is a sensor array.

### CROSS REFERENCE TO RELATED APPLICATIONS

This application is a U.S. National Phase application under 35 U.S.C. § 371 of International Application No. PCT/EP2020/086393 filed on Dec. 16, 2020, and claims benefit to German Patent Application No. DE 10 2020 101 132.6, filed on Jan. 20, 2020. The International Application was published in German on Jul. 29, 2021 as WO 2021/148203 A1 under PCT Article 21(2).

### FIELD

The present disclosure relates to an apparatus and a method for detecting multiple gases in high-voltage devices which are filled with insulating medium, in particular high-voltage transformers.

### BACKGROUND

WO 2012/120113 A1 has disclosed a system and a method for monitoring gases in power transformers which are cooled with oil. The system consists in this case of a bar and a main housing. The bar has two lines running in the interior and is positioned in the oil of the power transformer. The lines are connected to one another via two oil chambers and a pump such that the oil can be sucked in from the power transformer via a line to the first oil chamber and subsequently conducted back into the power transformer through the other line via the second oil chamber. The pump is in this case arranged in a line section between the oil chambers. An additional region with a temperature sensor and with a moisture sensor is situated before the pump. Both oil chambers have a wall which consists of a semipermeable basic material. Gases contained in the oil of the power transformer can migrate into the interior of the main housing through the wall. An additional gas sensor detects the gases which accumulate in the main housing of the system. Additionally, two valves having in each case one filter are arranged at the housing. One of the valves is used for sucking-in of air from the surroundings by means of a pump. The air is released from the interior of the main housing by way of the second valve. The system is controlled via a controller.

The above-described system is of highly complex construction. The multiplicity of individual parts used makes the system not only expensive, but also maintenance-intensive. The valves for the exchange of air wear particularly quickly and thus constitute a weakness in the system. The flat configured membrane can rupture particularly quickly in the event of a sudden pressure increase and in particular during the evaluation of the carrier gas.

### SUMMARY

In an embodiment, the present disclosure provides an apparatus that detects gas in a high-voltage device, which is filled with an insulating medium. The apparatus has: an inlet configured for introducing a carrier gas; an outlet configured for discharging the carrier gas; at least one gas sensor configured to detect a gas; a first pump configured to convey the carrier gas in the apparatus; a membrane which comprises at least one semipermeable basic material, which is at least partially surrounded by the insulating medium, and which is arranged to be at least partially subjected to an incident flow of the carrier gas; a second pump configured to convey the carrier gas into the apparatus and out of the apparatus; and a separating column, which is arranged before the gas sensor. The gas sensor is a sensor array.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a first embodiment of an apparatus for detecting gas;

FIG. 2 shows a first operating state of the first embodiment of an apparatus for detecting gas;

FIG. 3 shows a second operating state of the first embodiment of an apparatus for detecting gas;

FIG. 4 shows a second embodiment of an apparatus for detecting gas;

FIG. 5 shows a first operating state of the second embodiment of an apparatus for detecting gas;

FIG. 6 shows a second operating state of the second embodiment of an apparatus for detecting gas; and

FIG. 7 shows a method for detecting gas.

### DETAILED DESCRIPTION

Aspects of the present disclosure provide an apparatus for detecting gas molecules in a high-voltage device filled with insulating medium that is of inexpensive, maintenance-friendly and substantially wear-free construction, and provide a method for detecting gases in high-voltage devices filled with liquids that ensures reliable and accurate operation of the apparatus.

According to a first aspect, the present disclosure provides an apparatus for detecting multiple gases in a high-voltage device which is filled with an insulating medium, having:

an inlet for introducing and an outlet for discharging a carrier gas;

at least one gas sensor for detecting a gas;

a first pump for conveying the carrier gas in the apparatus;

a membrane which consists at least of at least one semipermeable basic material, is at least partially surrounded by the insulating medium, and is at least partially subjected to an incident flow of the carrier gas;

a second pump for conveying the carrier gas into the apparatus and for conveying it out of the apparatus;

characterized in that:

a separating column is provided and is arranged before the gas sensor, and the gas sensor is designed as a sensor array.

One advantage provided by aspects of the present disclousre is that the extension of the apparatus to include the combination of a separating column and a sensor array is both particularly expedient and particularly accurate. Sensor arrays are multiple particularly compact semiconductor sensors which are arranged on a unit. In this way, a measurement chamber can be of particularly compact design. Furthermore, sensor arrays are significantly more expedient in comparison with conventional sensors from the area of gas-in-oil analysis. The arrangement of a separating column before the sensor array makes possible pre-separation of the gases and thus improves the selectivity of the overall apparatus. Separating columns are commonly used in laboratory setups, although not in apparatuses directly at high-voltage devices. Furthermore, the separating column allows the number of individual sensors in the sensor array to be reduced. The measurements are simplified, which minimizes the computational outlay in the case of the data processing. Consequently, the central control device can be constructed with the simplest means, which has a positive effect on the costs of the apparatus. This combination leads overall to an inexpensive, robust, low-maintenance apparatus that is less susceptible to faults. A further advantage is that what is circulated is the carrier gas and not the insulating medium. In this way, it is possible for fresh carrier gas, such as for example ambient air, to be introduced into the apparatus before the enrichment and detection and to be discharged from the apparatus after the detection.

The sensor array may be designed in any desired manner according to requirement, for example as a semiconductor sensor consisting of oxides of metals or transition metals, such as for example stannic oxide (SnO2) or tungsten oxide (WO3), and/or doped with noble metals, such as for example platinum (Pt) or palladium (Pd). The sensor array may, for example, be constructed on the basis of the pellistor principle a/or be a Taguchi gas sensor.

The separating column may be designed in any desired manner according to requirement, for example either from metal or from fused silica. Internally, it may be lined with a defined stationary phase, for example with viscous polyorganosiloxanes. The length of the separating column is preferably approximately 0.5 to 1.0 m. The separating column may be of the column type PoraBond Q, WP Plot Q, Hayesep N or Hayesep Q.

The high-voltage device may be designed in any desired manner according to requirement, for example as a high-voltage transformer, power transformer, on-load tap-changer, circuit breaker, condenser bushing or some other oil-filled piece of electrical equipment.

The insulating medium may be of any desired type according to requirement, for example an insulating oil or ester liquid.

The gas to be detected may be of any desired type according to requirement and, for example, contain at least one hydrocarbon compound and/or other gas molecules and/or other gas atoms.

The semipermeable membrane may be of any desired form according to requirement and, for example, at least partially consist of Teflon.

The pumps may be designed in any desired manner according to requirement, for example as membrane pumps.

It may be provided that:

a valve having at least two operating states is provided.

The valve may be designed in any desired manner according to requirement, for example as a Valco 6-port valve or else as a Valco 8-port valve. It is preferably possible for the valve to be switched into at least two operating states.

It may be provided that:

an inlet, a first line and a filter are provided, and the first line is connected to a sixth connection of the valve;

the membrane is connected at one side to a first connection of the valve via a first connecting line and at the other side to the second connection of the valve via a second connecting line;

the first pump is arranged in the first connecting line;

a measurement chamber in which there are arranged sensors for temperature, moisture and pressure and/or gas sensors is provided;

a fourth connecting line connects the separating column to the measurement chamber via a fourth connection of the valve;

the measurement chamber is connected to an outlet via a second line.

It may be provided that

a sample loop is connected to a third connection of the valve via a third connecting line and to a fifth connection of the valve via a fourth connecting line.

The sample loop may be of any desired form according to requirement and, for example, at least partially consist of Teflon.

It may be provided that:

the sensors in the measurement chamber, the first pump, the second pump and the valve are connected to a control device; and

the sensors in the measurement chamber, the first pump, the second pump and the valve are controlled on the basis of the measurements of the sensors in the measurement chamber.

It may be provided that:

the inlet has a first line, a pump and a filter.

It may be provided that:

the membrane is of at least partially spiral-shaped form and/or of at least partially meandering form and/or of at least partially helical form.

It may be provided that:

at least one temperature sensor is provided.

It may be provided that:

the outlet has a second line.

It may be provided that:

a line or connecting line for transport of the gases to be analyzed, which line or collecting line has a pump, is present.

It may be provided that:

a measurement chamber in which there are arranged sensors for determining temperature, ambient moisture, pressure and different gases is provided.

It may be provided that:

at least one thermal element is arranged in the measurement chamber.

It may be provided that:

the temperature sensor and the thermal element are connected to a control device, and

the thermal element is controlled on the basis of the measurements of the temperature sensor.

It may be provided that:

during the operation of the first pump, the second pump functions as a closed valve, and during the operation of the second pump, the first pump functions as a closed valve.

It may be provided that:

the thermal element is in the form of a Peltier element.

It may be provided that:

the measurement chamber is lined internally with an inert material, platinum or gold or some other noble metal.

According to a second aspect, the present disclosure provides a method for detecting multiple gases in a high-voltage device, which is filled with an insulating medium, by means of an apparatus as described above, wherein:

in a first step, a measurement chamber is flushed with a carrier gas in that the carrier gas is conveyed into the measurement chamber through the separating column and conveyed out of the measurement chamber;

in a second step, the carrier gas is conveyed through a membrane and is enriched by gas which flows through the membrane;

in a third step, a sensor in the measurement chamber measures the gas after it has passed through the separating column.

It may be provided that:

in the first and third steps, the valve is in a first operating state for flushing of the measurement chamber and for measurement of the gases;

in the third step, the valve is in a second operating state for enrichment of the carrier gas.

It may be provided that:

the carrier gas is transported into the measurement chamber through a valve, a sample loop, the fourth connecting line and the separating column, and, at the measurement chamber, the gases collected in the membrane are detected by the sensors.

It may be provided that:

during the flushing, the carrier gas is sucked in through a first line, is conveyed into the measurement chamber through the valve and via the separating column, and is discharged through the outlet.

It may be provided that:

the flushing of the measurement chamber is realized by means of the second pump.

It may be provided that:

the conveyance of the carrier gas is realized by means of a first pump.

It may be provided that:

the amount and/or the type of the gas in the measurement chamber are/is determined before and/or after the carrier gas has been conveyed out.

It may be provided that:

the amount and/or the type of the gas in the measurement chamber are/is determined before and/or after the carrier gas has been conveyed.

Identical reference signs are used for identical or identically acting elements of the present disclosure. Furthermore, for the sake of clarity, in the individual figures, only reference signs necessary for the description of the respective figure are illustrated. The illustrated embodiments constitute merely examples of the configuration of the apparatus according to the present disclosure, and thus do not constitute a conclusive delimitation of the present disclosure.

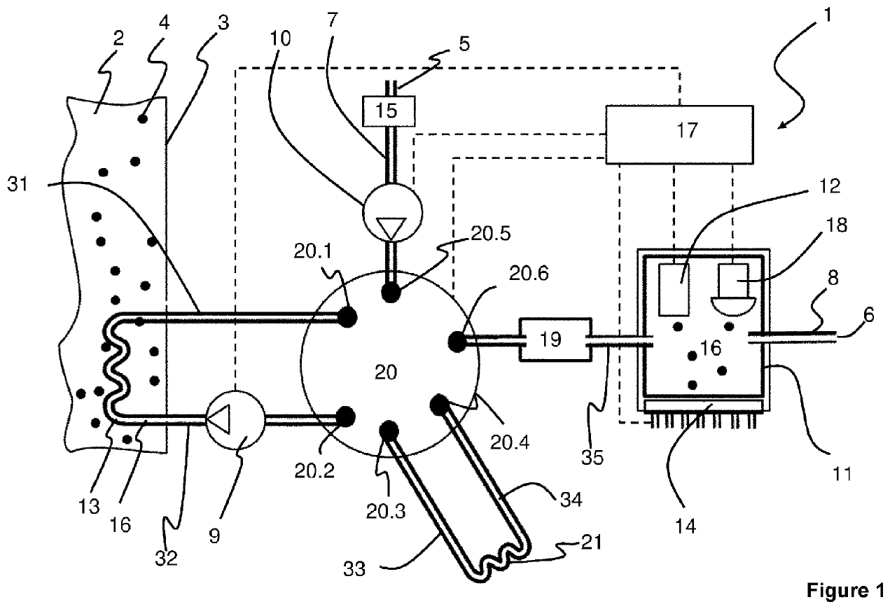

FIG. 1 shows, in a schematic illustration, a first embodiment of an apparatus 1 for detecting gas molecules, ions or gases 4 in a high-voltage device 3 which is filled with a liquid or an insulating medium 2. The high-voltage device 3 may be designed as a high-voltage transformer, power transformer, on-load tap-changer, circuit breaker or condenser bushing.

The apparatus 1 has a membrane or capillary 13 that consists of at least one semipermeable basic material and that is of tube-like or hose-like construction. The tube-like membrane 13 may be shaped in any desired manner, for example so as to be spiral-shaped and/or helical and/or meandering. Due to this advantageous configuration of the membrane 13, it is suitable for particularly high pressures. The membrane 13 is situated in the high-voltage device 3 or at least in a part of the high-voltage device with accessibility in relation to the insulating medium 2. Consequently, the membrane 13 may be arranged in a Buchholz relay, in a line of the cooling means, etc. Due to the tube-like configuration and the basic material which is gas-permeable (semipermeable) in one direction, molecules of the gas 4 can pass into a circuit of the apparatus 1.

The membrane 13 is connected at one end, which forms the entry of the membrane, to a first connection 20.1 of a valve 20 via a first connecting line 31 and, at the other side, at another end, which forms the exit of the membrane, to the second connection 20.2 of the valve 20 via a second connecting line 32. A first pump 9 is arranged in the second line 32.

The apparatus furthermore has a sample loop 21. A sample loop is a gas-tight capillary with a defined volume. This is normally produced from fused silica. However, it may also consist of a suitable plastic. The sample loop 21 is connected to the third and fourth connections 20.3, 20.4 of the valve 20 via a third and a fourth connecting line 33, 34. The apparatus 1 furthermore has a first line 7, which, at one side, has an inlet 5 and, at the other side, is connected to a fifth connection 20.5 of the valve 20. A filter 15 and a second pump 10 are arranged in the first line 7.

A separating column 19 is arranged between the sixth connection 20.6 and the measurement chamber 11 via a fifth connecting line 35. The measurement chamber 11 is moreover connected to an outlet 6 via a second line 8.

The measurement chamber 11 has a thermal element 14, for example a Peltier element, with the aid of which temperature control of the measurement chamber 11 is carried out. In addition, at least one gas sensor 12 and one temperature sensor 18 are arranged in the measurement chamber 11.

The gas sensor 12 is designed as a sensor array. The sensor array consists of multiple semiconductor sensors. The semiconductor sensors consist of oxides of metals or transition metals, such as for example stannic oxide (SnO2) or tungsten oxide (WO3), and, for improvement of selectivity, are doped with noble metals, such as for example platinum (Pt) or palladium (Pd). The sensor array is constructed on the basis of the pellistor principle, although this does not constitute the only design. Taguchi gas sensors are classical semiconductor sensors.

The measurement chamber 11 is lined or coated internally with an inert material, such as for example gold. This coating offers the advantage that the gases 4 cannot, in the interior, be deposited or condense and non-reproducibly taken up, in the process enter into at least polar physical bonding and thus be absent in the overall gas balance, whereby incorrect values would be measured in comparison with a laboratory analysis.

The separating column 19 may consist either of metal or of fused silica. Internally, it is lined with a defined stationary phase, for example with viscous polyorganosiloxanes. The length of the separating column is approximately 0.5-1.0 m. Common column types are PoraBond Q, WP Plot Q, Hayesep N and Hayesep Q.

The valve 20 has different operating states/positions. In the case of these, the individual connections are internally connected in such a way that different circuits or connections of individual components among one another are formed in the apparatus 1.

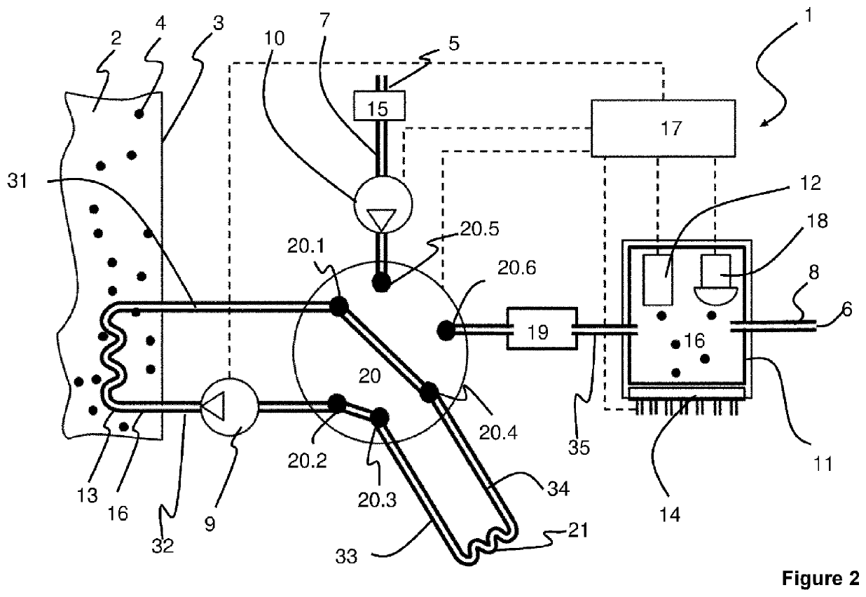

In a first operating state, as is illustrated in FIG. 2, for example a circuit for enrichment of the carrier gas 16 is produced. Here, the first and fourth connections 20.1, 20.4 and the second and third connections 20.2, 20.3 are internally connected. The first pump 9 conveys the carrier gas 16 through the second connecting line 32, the capillary 13 and the sample loop 21. Since the pressure in the interior of the high-pressure device 3 is at all times higher than the pressure of the surroundings and thus also the pressure in the apparatus 1, the gases 4 released in the insulating medium 2 pass into the circuit of the apparatus 1 through the semipermeable membrane 13. The carrier gas 16 is enriched through repeated conveyance or circulation. The valve 20 is likewise connected to the central control device 17. Air from the surroundings may be used as carrier gas 16.

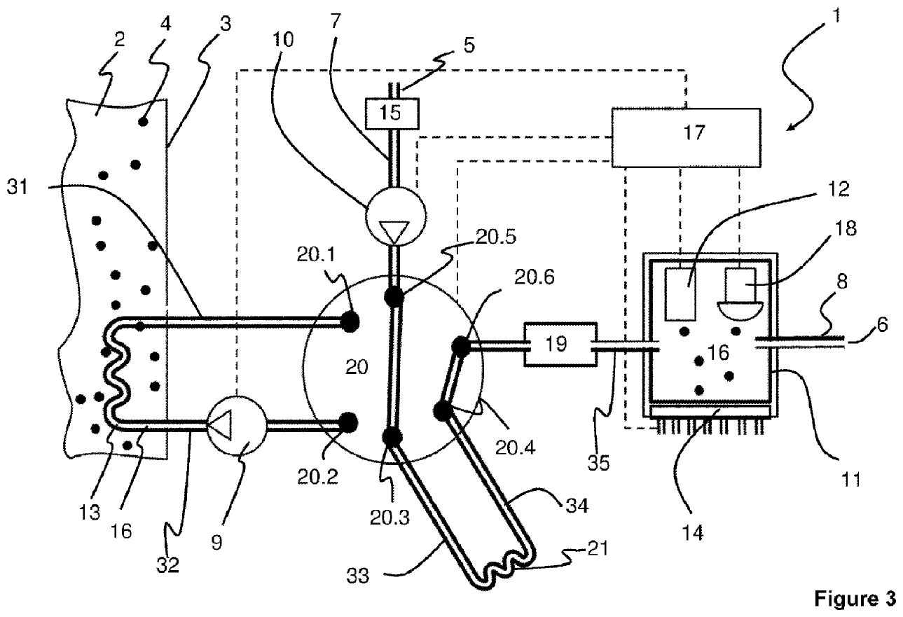

FIG. 3 illustrates a second operating state of the valve 20. Here, the fifth and third connections 20.5, 20.3 and also the fourth and sixth connections 20.4, 20,6 of the valve 20 are internally connected to one another. Fresh carrier gas 16 (for example ambient air) is conveyed into the apparatus 1 by means of second pump 10 via the inlet 5 of the first line 7. The carrier gas 16 situated in the sample loop 21 is introduced firstly into the separating column 19 and then into the measurement chamber 12. Subsequently, the carrier gas 16 and the sucked-in air are conveyed to the outlet 6 through the second line 8. The gas sensor 12, the temperature sensor 18 and the thermal element 14 in the measurement chamber 11 and also the first and second pumps 9, 10 are connected to a central control device 17. The control of the thermal element 14 is realized on the basis of the measurements of the temperature sensor 18.

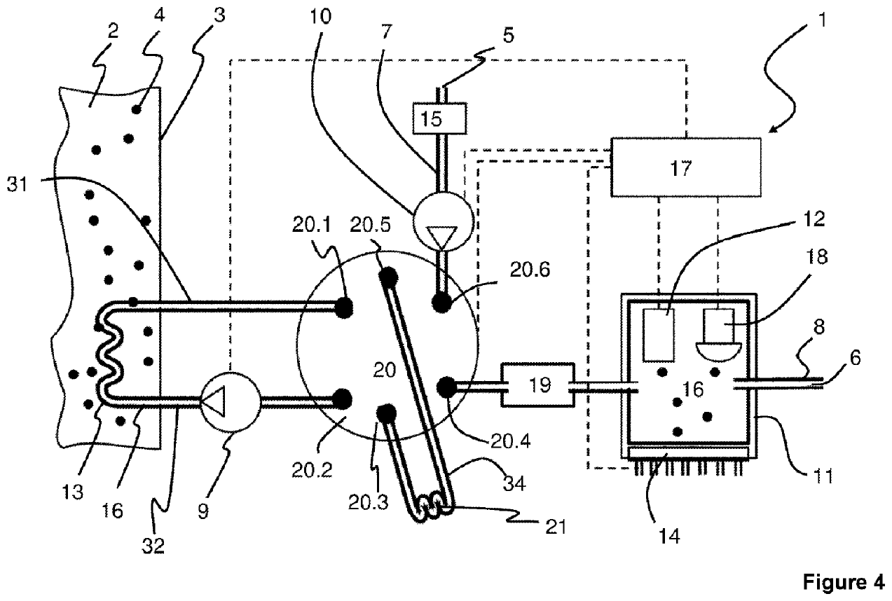

The valve 20 may preferably be designed as a Valco 6-port valve or else as a Valco 8-port valve. The valve may assume two operating states internally. With the use of a Valco 6-port valve, the individual parts of the apparatus 1 are connected to the valve 20 in a specific way. This is illustrated in FIG. 4. Here, it is only ever possible for in each case two adjacent connections of the valve to be internally connected to one another. In this way, the valve can be actuated in a particularly expedient and simple manner. The membrane 13 is connected at one end, which forms the entry of the membrane, to a first connection 20.1 of a valve 20 via a first connecting line 31 and, at the other side, at another end, which forms the exit of the membrane, to the second connection 20.2 of the valve 20 via a second connecting line 32. A first pump 9 is arranged in the second line 32.

The apparatus furthermore has a sample loop 21. The sample loop 21 is connected to the third and fifth connections 20.3, 20.5 of the valve 20 via a third and a fourth connecting line 33, 34. The apparatus 1 furthermore has a first line 7, which, at one side, has an inlet 5 and, at the other side, is connected by way of a sixth connection 20.6 to the valve 20. A filter 15 and a second pump 10 are arranged in the first line 7.

A separating column 19 is arranged between the fourth connection 20.4 and the measurement chamber 11 via a fifth connecting line 35. The measurement chamber 11 is moreover connected to an outlet 6 via a second line 8.

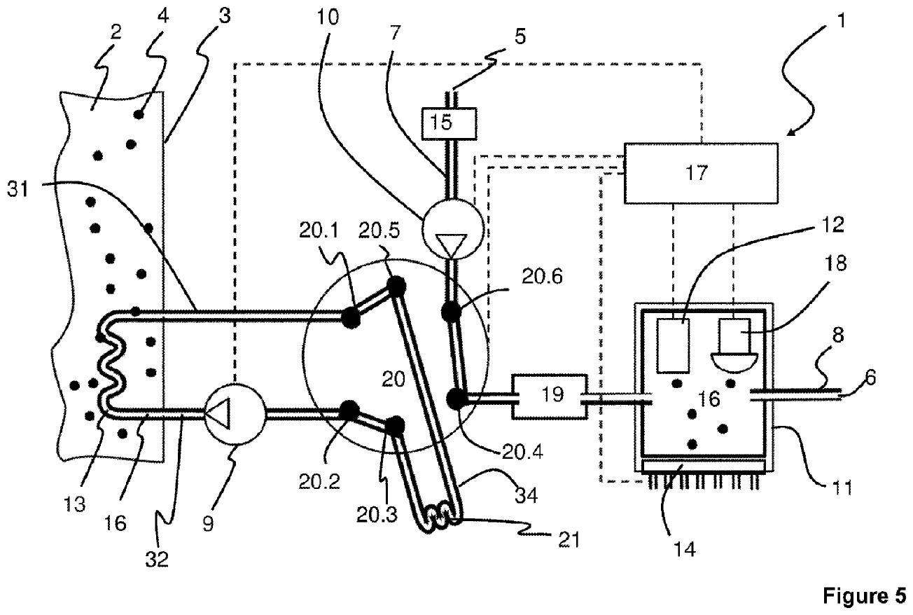

In a first operating state, as is illustrated in FIG. 5, for example a circuit for enrichment of the carrier gas 16 is produced. Here, the first and fifth connections 20.1, 20.5 and the second and third connections 20.2, 20.3 are internally connected. The fifth and fourth connections 20.4, 20.5 are likewise connected to one another. This is unimportant for the enrichment. The first pump 9 conveys the carrier gas 16 through the second connecting line 32, the capillary 13 and the sample loop 21. Since the pressure in the interior of the high-pressure device 3 is at all times higher than the pressure of the surroundings and thus also the pressure in the apparatus 1, the gases 4 released in the insulating medium 2 pass into the circuit of the apparatus 1 through the semipermeable membrane 13. The carrier gas 16 is enriched through repeated conveyance or circulation. The valve 20 is likewise connected to the central control device 17. Air from the surroundings may be used as carrier gas 16.

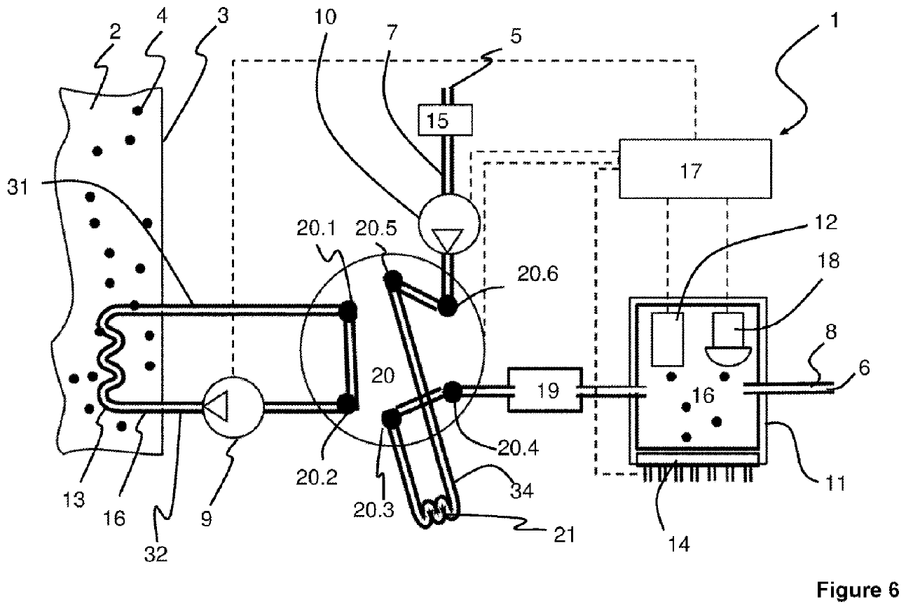

FIG. 6 illustrates a second operating state of the valve 20. Here, the fifth and fourth connections 20.3, 20.4 and also the fifth and sixth connections 20.5, 20.6 of the valve 20 are internally connected to one another. Fresh carrier gas 16 (for example ambient air) is conveyed into the apparatus 1 by means of second pump 10 via the inlet 5 of the first line 7. The carrier gas 16 situated in the sample loop is introduced firstly into the separating column 19 and then into the measurement chamber 12. Subsequently, the carrier gas 16 and the sucked-in air are conveyed to the outlet 6 through the second line 8.

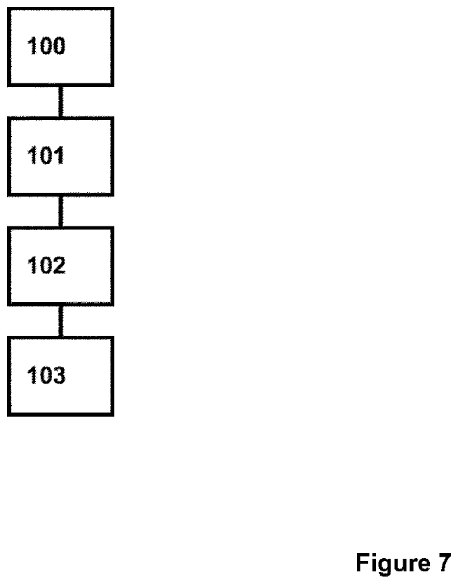

FIG. 7 shows a flow diagram for a method for detecting gases in a high-voltage device 3, which is filled with a liquid 2, by means of the apparatus 1 that comprises the following steps:

Step 100: Firstly, the apparatus 1 is flushed. In this case, the valve 20 is in the second operating state. The first pump 9 is in the switched-off state, while the second pump 10 is in the switched-on state. The carrier gas 16, for example ambient air, is sucked in via the inlet 5, that is to say the filter 15 and the first line 7. In this case, the carrier gas 16 passes through the filter 15, the valve 20, the sample loop 21, the separating column 19 and the measurement chamber 11 before it passes to the outlet 6. The apparatus 1 is flushed in this manner.

Step 101: The flushing is ended after a predetermined time or on the basis of the measurements of the gas sensors 12 in the measurement chamber 11. The parameters ascertained in the measurement chamber 11 at the conclusion serve as starting point or zero point for the further measurements.

Step 102: In the enrichment phase of the embodiments in FIGS. 3 and 6, the valve 20 is switched into the first operating state and the first pump 9 is switched on, whereby the carrier gas 16 in the apparatus 1 is conveyed or circulated. The second pump 10 is in the switched-off state. The carrier gas 16 is moved in a circuit between the sample loop 21 and the membrane 13. Since the pressure in the interior of the high-pressure device 3 is higher than the pressure in the apparatus 1, gases 4 pass into the apparatus 1 from the insulating medium 2 through the membrane 13, which is permeable to gas molecules in one direction. This results in enrichment of the carrier gas 16. The duration of the enrichment may be configured to be variable.

Step 103: After the enrichment phase, the switching of the valve 20 into the second operating state is realized. The enriched gas in the sample loop 21 is transported with the carrier gas 16 into the measurement chamber 11 through the separating column 19, and the amount and the type of the gases 4 in the measurement chamber 11 are determined via the gas sensor 12. After the determination of the gases 4, the apparatus 1 is flushed by way of the continued sucking-in of fresh ambient air.

The method described may be carried out either permanently or else a few times a day. Discontinuous operation of the apparatus 1 can lead to an increase in the service life of the gas sensors 12 used in the measurement chamber 11.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

1 Apparatus

2 Insulating medium

3 High-voltage device

4 Gas

5 Inlet

6 Outlet

7 First line

8 Second line

9 First pump

10 Second pump

11 Measurement chamber

12 Gas sensor

13 Membrane

14 Thermal element

15 Filter

16 Carrier gas

17 Control device

18 Temperature sensor

19 Separating column

20 Distributor/valve/switching valve

20.1 First connection

20.2 Second connection

20.3 Third connection

20.4 Fourth connection

20.5 Fifth connection

20.6 Sixth connection

21 Sample loop

31 First connecting line

32 Second connecting line

33 Third connecting line

34 Fourth connecting line

35 Fifth connecting line

1. An apparatus for detecting gas in a high-voltage device which is filled with an insulating medium, the apparatus comprising:
an inlet configured for introducing a carrier gas;
an outlet configured for discharging a-the carrier gas;
at least one gas sensor configured to detect a gas;
a first pump for conveyin configured to convey the carrier gas in the apparatus;
a membrane which comprises at least one semipermeable basic material, which is at least partially surrounded by the insulating medium, and which is arranged to be at least partially subjected to an incident flow of the carrier gas;
a second pump configured to convey the carrier gas into the apparatus and out of the apparatus; and
a separating column, which is arranged before the gas sensor,
wherein the gas sensor is as a sensor array.

2. The apparatus as claimed in claim 1, wherein
the membrane is at least partially of a tube-like construction or at least partially of a hose-like construction.

3. The apparatus as claimed in claim 1, the apparatus further comprising:
a valve having at least two operating states.

4. The apparatus as claimed in claim 1, the apparatus further comprising:
an inlet, a first line and a filter,
wherein the first line is connected to a sixth connection of a valve.

5. The apparatus as claimed in claim 1, wherein:
the membrane is connected at one side to a first connection of a valve via a first connecting line and at the other side to a second connection of the valve via a second connecting line; and
the first pump is arranged in the first connecting line.

6. The apparatus as claimed in claim 1, the apparatus further comprising:
a measurement chamber in which there are arranged sensors for temperature, moisture and pressure, or gas sensors;
a fourth connecting line that connects the separating column to the measurement chamber via a fourth connection of a valve; and
the measurement chamber is connected to an outlet via a second line.

7. The apparatus as claimed in claim 1, the apparatus further comprising:
a sample loop connected to a third connection of a valve via a third connecting line and to a fifth connection of the valve via a fourth connecting line;

8. The apparatus as claimed in claim 6, wherein:
the sensors in the measurement chamber, the first pump, the second pump and the valve are connected to a controller; and
the sensors in the measurement chamber, the first pump, the second pump and the valve are configured to be controlled on a basis of the measurements of the sensors in the measurement chamber.

9. A method for detecting gas in a high-voltage device which is filled with an insulating medium, the method comprising:
in a first step, a measurement chamber is flushed with a carrier gas such that the carrier gas is conveyed into the measurement chamber through a separating column and conveyed out of the measurement chamber;
in a second step, the carrier gas is conveyed through a membrane and is enriched by gas which flows through the membrane;
in a third step, a sensor in the measurement chamber measures the gas after the gas has passed through the separating column.

10. The method as claimed in claim 9, wherein:
in the first step and the third step, the valve is in a first operating state for flushing of the measurement chamber and for measurement of the gases;
in the third step, the valve is in a second operating state for enrichment of the carrier gas.

11. The method as claimed in claim 9, wherein:
the carrier gas is transported into the measurement chamber through a valve, a sample loop, a fourth connecting line and the separating column, and, at the measurement chamber, gases collected in the membrane are detected by the sensors.

12. The method as claimed in claim 9, wherein:
during the flushing, the carrier gas is sucked in through a first line, is conveyed into the measurement chamber through the valve and via the separating column, and is discharged through the outlet.

13. The method as claimed in claim 9, wherein:
lithe flushing of the measurement chamber is realized using a second pump.

14. The method as claimed in claim 9, wherein
the conveyance of the carrier gas is realized by using a first pump.

15. The method as claimed in claim 9, wherein:
the an amount and/or a type of the gas in the measurement chamber are/is determined before and/or after the carrier gas has been conveyed out.
